(19) United States
(12) Patent Application Publication
(10) Pub. No.: US 2011/0230555 A1
(43) Pub. Date: Sep. 22, 2011

(54) Enzyme Inhibitors and the Use Thereof

(76) Inventors: David Middlemiss, Hertfordshire (GB); Kenneth J. Ingold, Durham, NC (US)

(21) Appl. No.: 12/999,732

(22) PCT Filed: Jun. 24, 2009
(86) PCT No.: PCT/US09/03757

§ 371 Date: Mar. 10, 2011

Related U.S. Application Data
(60) Provisional application No. 61/075,098, filed on Jun. 24, 2008.

(51) Int. Cl. A61K 31/216; C07C 229/26; C12N 9/99; A61P 3/10; A61P 3/04; A61P 9/04; A61P 9/10; A61P 9/12; A61P 3/06; A61P 5/50; A61P 17/00; A61P 17/06; A61P 35/00; A61K 31/22

(52) U.S. Cl. 514/542; 560/34; 435/184; 560/169; 514/551

(57) ABSTRACT

The present invention provides compounds and methods for the treatment of diseases or disorders such as heart failure, hyperlipidemia, hypercholesterolemia, gonadotropin deficiency, diabetes mellitus, metabolic syndrome, hyperglycemia, insulin resistance, glucose intolerance, obesity, psoriasis, atopic dermatitis, and cancer.

### FIELD OF INVENTION

The present invention is directed to compounds that inhibit carnitine palmitoyl transferase. The invention also relates to pharmaceutical compositions comprising the compounds and use of the compounds in the treatment of diseases or disorders associated with carnitine palmitoyl transferase.

### BACKGROUND OF THE INVENTION

Carnitine palmitoyl transferase (CPT) is a family of membrane bound long-chain acylcarnitine transferases found in several organs and is localized in subcellular organelles, such as mitochondria. CPT1 is localized on the outer mitochondrial membrane and catalyzes the formation of long-chain acylcarnitines, such as palmitoyl carnitine. Three tissue specific isoforms of CPT1 have been identified in the liver, brain, and muscle. Once CPT1 has catalyzed the formation of long-chain acylcarnitines, they are transported across the mitochondrial membrane by the inner mitochondrial membrane protein carnitine-acylcarnitine translocase. CPT2, localized on the inner mitochondrial membrane, catalyzes the conversion of long-chain acylcarnitines into long-chain acyl-coenzyme A esters, which are then oxidized into acetyl-coenzyme A in the mitochondrial matrix. Acetyl-coenzyme A activates pyruvate carboxylase, a key enzyme in the gluconeogenic pathway.

It has been reported that diabetic patients have high blood levels of fatty acids. These fatty acids are oxidized in the liver to produce an abundance of acetyl-coenzyme A, ATP, and NADH, which results in over-stimulation of the gluconeogenic pathway and increased blood glucose levels. Thus, inhibition of CPT1 would decrease the amount of acetyl-coenzyme A and consequently reduce gluconeogenesis and blood glucose levels.

U.S. Pat. Nos. 6,444,701 and 6,369,073, International Patent Publications WO 2006/092204 and WO 2008/015081 and Giannessi et al. (J. Med. Chem. 44:2382 (2001)) disclose a number of aminocarnitine derivatives that are inhibitors of CPT1. However, there still remains a need in the art for compounds that more effectively inhibit CPT1 and methods for treating diseases associated with CPT.

### SUMMARY OF THE INVENTION

The present invention relates to compounds which are inhibitors of CPT1, having the following Formula 1:

RO(CH₂)ₘX (I)

wherein X is selected from the group consisting of
NHCONHCH(CH₂CO₂)⁻CH₂N(CH₃)₃⁺,
NHCONHCH(CH₂CO₂H)CH₂N(CH₃)₃⁺Y⁻, and
NHCONHCH(CH₂CO₂R¹)CH₂N(CH₃)₃⁺Y;
R is selected from the group consisting of CH₃(CH₂)ₙ, PhC₆H₄(CH₂)ₚ, and Ph(CH₂)_{q};
R¹ is a C₁₋₄ straight or branched alkyl group;
Y⁻ is an anion;
m is 3 to 14;
n is 0 to 11;
p is 0 to 6;
q is 1 to 9;
wherein m plus n is 10 to 14;
m plus p is 5 to 9; and
m plus q is 8 to 12;
or pharmaceutically acceptable salts, prodrugs, or stereoisomers thereof.

In one embodiment of the compounds of Formula I, R is CH₃(CH₂)ₙ, m is 3 to 14, n is 0 to 11, and m plus n is 10 to 14. In another embodiment, R is PhC₆H₄(CH₂)ₚ, m is 3 to 9, p is 0 to 6, and m plus p is 5 to 9. In a further embodiment, R is Ph(CH₂)_{q}, m is 3 to 12, q is 1 to 9, and m plus q is 8 to 12.

The invention further relates to compounds which are inhibitors of CPT1, having the following Formula II:

R²-biphenyl(CH₂)ᵥX (II)

wherein X is selected from the group consisting of
NHCONHCH(CH₂CO₂)⁻CH₂N(CH₃)₃⁺,
NHCONHCH(CH₂CO₂H)CH₂N(CH₃)₃⁺Y⁻, and
NHCONHCH(CH₂CO₂R¹)CH₂N(CH₃)₃⁺Y⁻;
R¹ is a C₁₋₄ straight or branched alkyl group;
Y⁻ is an anion;
R² is selected from the group consisting of H and CH₃(CH₂)_{w};
v is 2 to 10;
w is 0 to 7; and
v plus w is 5 to 10;
or pharmaceutically acceptable salts, prodrugs, or stereoisomers thereof.

In one embodiment, R² is H and v is 6 to 10. In another embodiment, R² is CH₃(CH₂)_{w}, v is 2 to 9, w is 0 to 7, and v plus w is 5 to 9.

The invention further relates to pharmaceutical compositions comprising, consisting essentially of, or consisting of the compounds of the invention and a pharmaceutically acceptable carrier. As used herein, the term “consisting essentially of

As another aspect, the invention provides a method of inhibiting CPT1 comprising contacting the enzyme with a compound of the present invention. The enzyme can be located in an animal (e.g., a human), in an isolated cell or tissue, or in a solution.

In one aspect, the invention provides methods for treating diseases or disorders associated with CPT, such as diabetes, hyperglycemia, cancer, or psoriasis, by administering to an animal an effective amount of a compound of the present invention. In particular embodiments, the compound reduces the activity of CPT1, e.g., the activity of a liver isoform of CPT1 (CPT1L). In one embodiment, the compounds are administered topically.

In another aspect, the invention provides methods for treating diseases or disorders by administering to an animal an effective amount of a compound of the present invention.

The invention further relates to kits comprising the compounds and/or pharmaceutical compositions of the invention.

As still another aspect, the invention provides a method of preparing compounds of the present invention by reacting an isobutylcarnitine with a corresponding isocyanate to form an aminocarnitine-derived urea ester, then hydrolyzing the ester group of the aminocarnitine-derived urea ester to form a compound having general Formula I or II.

One embodiment of the invention relates to the use of the compounds of the invention in the preparation of a medicament for the inhibition of CPT1. Another embodiment of the invention relates to the use of the compounds of the invention in the preparation of a medicament for the treatment of diseases or disorders associated with CPT. One embodiment of the invention relates to the use of the compounds of the invention for the inhibition of CPT1. Another embodiment of the invention relates to the use of the compounds of the for the treatment of diseases or disorders associated with CPT.

### DETAILED DESCRIPTION OF THE INVENTION

As used herein, “a,” “an,” or “the” can mean one or more than one. For example, “a” cell can mean a single cell or a multiplicity of cells.

Also as used herein, “and/or” refers to and encompasses any and all possible combinations of one or more of the associated listed items, as well as the lack of combinations when interpreted in the alternative (“or”).

Furthermore, the term “about,” as used herein when referring to a measurable value such as an amount of a compound or agent of this invention, dose, time, temperature, and the like, is meant to encompass variations of ±20%, ±10%, ±5%, ±1%, ±0.5%, or even ±0.1% of the specified amount.

The term “consists essentially of (and grammatical variants), as applied to a composition of this invention, means a composition that contains no additional elements that materially alter the composition.

The present invention relates to compounds which are inhibitors of CPT1, having the following Formula I:

RO(CH₂)ₘX (I)

wherein X is selected from the group consisting of
NHCONHCH(CH₂CO₂)⁻CH₂N(CH₃)₃⁺,
NHCONHCH(CH₂CO₂H)CH₂N(CH₃)₃⁺Y⁻, and
NHCONHCH(CH₂CO₂R¹)CH₂N(CH₃)₃⁺Y⁻;
R is selected from the group consisting of CH₃(CH₂)ₙ, PhC₆H₄(CH₂)ₚ, and Ph(CH₂)ₚ, and Ph(CH₂)_{q};
R¹ is a C₁ straight or branched alkyl group;
Y⁻ is an anion;
m is 3 to 14;
n is 0 to 11;
p is 0 to 6;
q is 1 to 9;
wherein m plus n is 10 to 14;
m plus p is 5 to 9; and
m plus q is 8 to 12;
or pharmaceutically acceptable salts, prodrugs, or stereoisomers thereof.

In one embodiment of the compounds of Formula I, R is CH₃(CH₂)ₙ, m is 3 to 14, n is 0 to 11, and m plus n is 10 to 14. In another embodiment, R is PhC₆H₄(CH₂)ₚ, m is 3 to 12, to 9, p is 0 to 6, and m plus p is 5 to 9. In a further embodiment, R is Ph(CH₂)_{q}, m is 3 to 12, q is 1 to 9, and m plus q is 8 to 12. In other embodiments, m is 3, 4, 5, 6, 7, 8, 9, 10, 11, 12, 13, or 14 or any range therein, n is 0, 1, 2, 3, 4, 5, 6, 7, 8, 9, 10, or 11 or any range therein, p is 0, 1, 2, 3, 4, 5, or 6 or any range therein, and q is 1, 2, 3, 4, 5, 6, 7, 8, or 9 or any range therein. In further embodiments, m plus n is 10, 11, 12, 13, or 14 or any range therein, m plus p is 5, 6, 7, 8, or 9 or any range therein, and m plus q is 8, 9, 10, 11, or 12, or any range therein.

Examples of compounds of Formula I include, without limitation: isobutyl
(R)-4-trimethylammonio-3-[3-[6-(4-phenylbutoxy)hex-1-yl]ureido]butyrate formate,
(R)-4-trimethylammonio-3-[3-[6-(4-phenylbutoxy)hex-1-yl]ureido]butyrate,
(R)-4-trimethylammonio-3-[3-[6-(2-phenylethoxy)hex-1-yl]ureido]butyrate,
isobutyl (R)-4-trimethylammonio-3-[3-(12-methoxydodec-1-yl)ureido]butyrate formate,
(R)-4-trimethylammonio-3-[3-(12-methoxydodec-1-yl)ureido]butyrate,
isobutyl (R)-4-trimethylammonio-3-[3-(6-heptyloxyhex-1-yl)ureido]butyrate formate,
(R)-4-trimethylammonio-3-[3-(6-heptyloxyhex-1-yl)ureido]butyrate,
isobutyl (R)-4-trimethylammonio-3-[3-[7-(4-biphenyloxy)hept-1-yl]ureido]butyrate formate, and
(R)-4-trimethylammonio-3-[3-7-(4-biphenyloxy)hept-1-yl]ureido]butyrate, as shown in Table 1.

In one embodiment of the compounds of Formula I, p is 1 to 6. In another embodiment, the compounds of Formula I excludes (R)-4-trimethylammonio-3-[3-(7-(4-biphenyloxy)heptyl)ureido]butyrate.

The invention further relates to compounds which are inhibitors of CPT1, having the following Formula II:

R²-biphenyl(CH₂)ᵥX (II)

wherein X is selected from the group consisting of
NHCONHCH(CH₂CO₂)⁻CH₂N(CH₃)₃⁺,
NHCONHCH(CH₂CO₂H)CH₂N(CH₃)₃⁺Y⁻, and
NHCONHCH(CH₂CO₂R¹)CH₂N(CH₃)₃⁺Y⁻;
R¹ is a C₁₋₄ straight or branched alkyl group;
Y⁻ is an anion;
R² is selected from the group consisting of H and CH₃(CH₂)_{w};
v is 2 to 10;
w is 0 to 7; and
v plus w is 5 to 10;
or pharmaceutically acceptable salts, prodrugs, or stereoisomers thereof.

In one embodiment, R² is H and v is 6 to 10. In another embodiment, R² is CH₃(CH₂)_{w}, v is 2 to 9, w is 0 to 7, and v plus w is 5 to 9. In further embodiments, v is 2, 3, 4, 5, 6, 7, 8, 9, or 10 or any range therein, w is 0, 1, 2, 3, 4, 5, 6, or 7 or any range therein, and v plus w is 5, 6, 7, 8, 9, or 10 or any range therein.

In one embodiment of the compounds of the invention, the R² group is attached to the biphenyl group at the para position with respect to the phenyl ring to which R² is not attached. In other embodiments, the R² group is attached to the biphenyl group at the ortho or meta position.

An example of compounds of Formula II includes, without limitation: isobutyl (R)-4-trimethylammonio-3-[3-[7-(4-biphenylyl)hept-1-yl]ureido]butyrate and (R)-4-trimethylammonio-3-[3-[7-(4-biphenylyl)hept-1-yl]ureido]butyrate, as shown in Table 1.

TABLE 1
Examples of compounds of Formula I and II
Structure

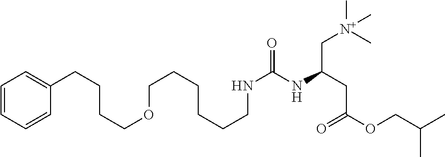

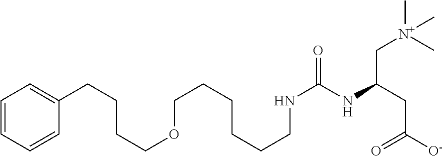

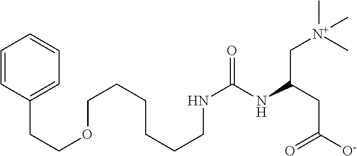

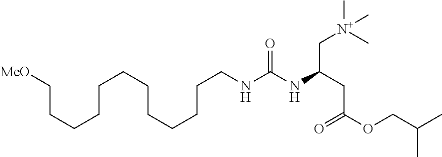

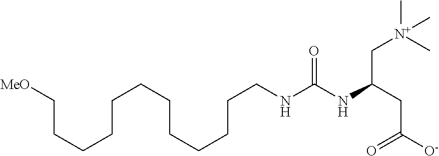

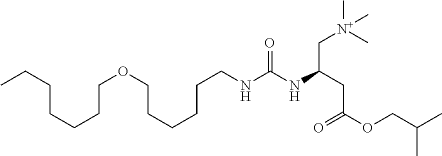

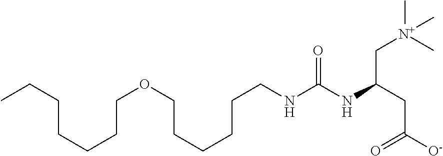

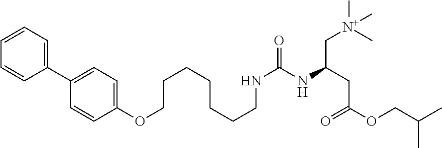

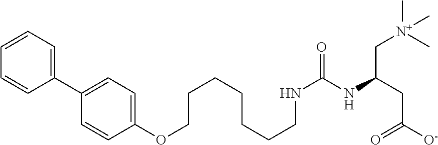

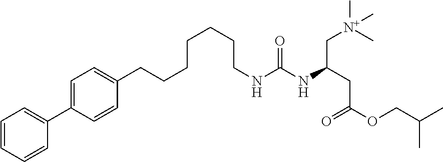

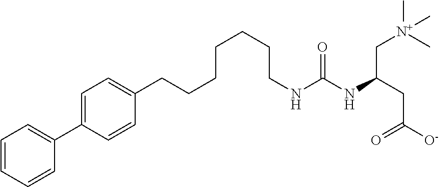

Examples of suitable R¹ alkyl groups include, without limitation, methyl, ethyl, propyl, isopropyl, butyl, isobutyl, tert-butyl, or sec-butyl.

Y¹ can be any suitable anion that forms a counterion for the compounds of the present invention as discussed below. In one embodiment, Y¹ is a pharmaceutically acceptable anion.

The present invention also comprises tautomers, geometrical isomers, optically active forms such as enantiomers and racemate forms of the compounds of Formula I and II. The compounds of Formula I and II have an asymmetry center on the carbon atom attached to the ureido group. For the purposes of the invention, each compound of Formula I and II can exist both as the R,S racemic mixture and as separated R and S isomeric forms.

The compounds of the present invention can be prodrugs that are converted to the active compound in vivo. The term “prodrug” denotes a compound which, upon administration to a subject, undergoes chemical conversion by metabolic or chemical processes to yield a compound of the present invention. The “prodrug” can be a compound of the present invention that has been chemically derivatized such that, (i) it retains some, all, or none of the bioactivity of its parent drug compound, and (ii) it is metabolized in a subject to yield the parent drug compound. For example, the compound optionally comprises a substituent that is convertible in vivo to a different substituent, such as hydrogen.

Prodrugs of the compounds of the present invention may be conventional esters. Examples of some common esters include but are not limited to phenyl esters, aliphatic esters (C₁-C₂₄), acyloxymethyl esters, carbamates, amino acid esters, and carboxylate esters where the group is alkyl, aryl, aralkyl, acyloxyalkyl, or alkoxycarbonyloxyalkyl. In one embodiment, the esters are C₁ to C₄ esters, e.g., methyl, ethyl, propyl, isopropyl, butyl, isobutyl, tert-butyl, or sec-butyl. The groups illustrated are exemplary, not exhaustive, and one skilled in the art could prepare other known varieties of prodrugs. Conventional procedures for the selection and preparation of suitable prodrug derivatives are described, for example, in “Design of Prodrugs,” ed. H. Bundgaard, Elsevier, 1985, which is incorporated by reference herein in its entirety.

In one embodiment, the compounds of the present invention may exist as inner salts, i.e., where X is NHCONHCH(CH₂CO₂)⁻CH₂N(CH₃)₃⁺. The term “inner salt” refers to a compound that exists as a zwitterion, meaning the compound carries both a negative and a positive charge. In other embodiments, the invention encompasses salts of the compounds described above that are not inner salts, i.e., when X is NHCONHCH(CH₂CO₂H)CH₂N(CH₃)₃⁺Y⁻ or NHCONHCH(CH₂CO₂R¹)CH₂N(CH₃)₃⁺Y⁻.

The term “pharmaceutically acceptable salts” refers to salts that retain the desired biological activity of the parent compound and do not impart undesired toxicological effects thereto.

Pharmaceutically acceptable base addition salts can be formed with metals or amines, such as alkali and alkaline earth metals or organic amines. Examples of metals used as cations are sodium, potassium, magnesium, calcium, and the like. Examples of suitable amines are N,N′-dibenzylethylenediamine, chloroprocaine, choline, diethanolamine, dicyclohexylamine, ethylenediamine, N-methylglucamine, and procaine (see, for example, Berge et al., “Pharmaceutical Salts,” J. of Pharm. Sci. 66:1 (1977)). The base addition salts of acidic compounds are prepared by contacting the free acid form with a sufficient amount of the desired base to produce the salt in the conventional manner. The free acid form may be regenerated by contacting the salt form with an acid and isolating the free acid in the conventional manner. The free acid forms differ from the respective salt forms somewhat in certain physical properties such as solubility in polar solvents, but otherwise the salts are equivalent to their respective free acid for purposes of the present invention. As used herein, a “pharmaceutical addition salt” includes a pharmaceutically acceptable salt of an acid form of one of the components of the compositions of the invention. These include organic or inorganic acid salts of the amines. Preferred acid salts are the hydrochlorides, acetates, salicylates, nitrates and phosphates. Other suitable pharmaceutically acceptable salts are well known to those skilled in the art and include basic salts of a variety of inorganic and organic acids including, for example, with inorganic acids, such as hydrochloric acid, hydrobromic acid, sulfuric acid or phosphoric acid; with organic acids such as carboxylic, sulfonic, sulfo or phospho acids or N-substituted sulfamic acids, for example acetic acid, propionic acid, glycolic acid, succinic acid, maleic acid, hydroxymaleic acid, methylmaleic acid, fumaric acid, mucic acid, malic acid, tartaric acid, lactic acid, oxalic acid, gluconic acid, glucaric acid, glucuronic acid, citric acid, benzoic acid, cinnamic acid, mandelic acid, salicylic acid, 4-aminosalicylic acid, 2-phenoxybenzoic acid, 2-acetoxybenzoic acid, embonic acid, nicotinic acid or isonicotinic acid; and with amino acids, such as naturally-occurring alpha-amino acids, for example glutamic acid or aspartic acid, and also with phenylacetic acid, methanesulfonic acid, ethanesulfonic acid, 2-hydroxyethanesulfonic acid, ethane-1,2-disulfonic acid, benzenesulfonic acid, 4-methylbenzenesulfonic acid, naphthalene-2-sulfonic acid, naphthalene-1,5-disulfonic acid, 2- or 3-phosphoglycerate, glucose-6-phosphate, N-cyclohexylsulfamic acid (with the formation of cyclamates), or with other acid organic compounds, such as ascorbic acid. Pharmaceutically acceptable salts of compounds may also be prepared with a pharmaceutically acceptable cation. Suitable pharmaceutically acceptable cations are well known to those skilled in the art and include alkaline, alkaline earth, ammonium and quaternary ammonium cations. Carbonates or hydrogen carbonates are also possible.

The compounds of the present invention may be prepared from readily available starting materials using the following general methods and procedures and as described in the examples. It will be appreciated that where typical or preferred experimental conditions (i.e., reaction temperatures, time, moles of reagent, solvents, etc.) are given, other experimental conditions can be used, unless otherwise stated. Optimum reaction conditions may vary with the particular reactants or solvents used, but such conditions can be determined by one skilled in the art by routine optimization procedures.

A process for preparing the compounds of the present invention comprises reacting an isobutylcarnitine, such as (R)-3-aminocarnitine isobutyl ester, with the corresponding isocyanate in a dipolar aprotic or protic solvent, such as isobutanol, at temperatures between about 4° C. and about 80° C., such as between about 10° C. and about 60° C., for times between about 1 and about 72 hours, such as between about 15 and about 48 hours, to yield an aminocarnitine-derived urea ester.

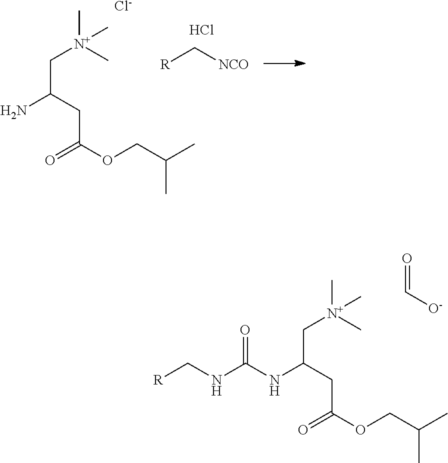

The isocyanates may be commercially available or produced by any known method, such as starting from the appropriate amine using triphosgene in the presence of DIPEA to produce the desired isocyanate. The amines may be commercially available or prepared by any known method, such as the reduction of a nitrile to produce the desired amine. Conversion of the aminocarnitine-derived urea esters to the acid compounds may be achieved by hydrolyzing the ester group of the aminocarnitine-derived urea ester under aqueous acidic or basic conditions at temperatures between about 10° C. and about 40° C., such as between about 10° C. and about 25° C., and for times between about 1 and about 120 hours, such as between about 15 and about 40 hours.

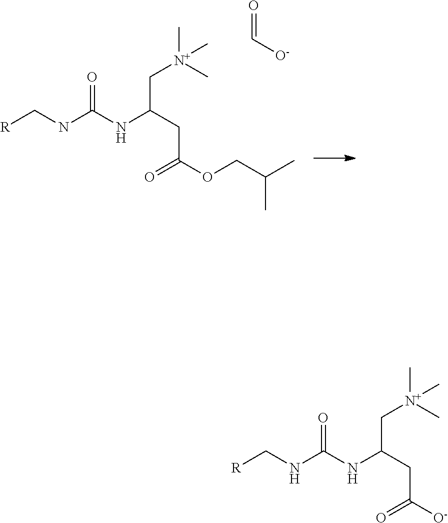

In one aspect the invention provides methods of inhibiting CPT1. The methods comprise contacting the enzyme with a compound of the present invention. The enzyme can be located in a cell in an animal (e.g., a human), in an isolated cell or tissue, or in a solution. The CPT1 may be any isoform of CPT1, including liver, brain, and/or muscle isoforms. The compound may preferentially inhibit one isoform or more than one isoform.

In another aspect, the methods of the present invention provide for the administration of an effective amount of a compound having Formula I or II to treat diseases or disorders associated with CPT. The of compound administered is effective to reduce the activity of CPT1 by at least about 10%, e.g., at least about 20%, 30%, 40%, 50%, 60%, 70%, 80%, 90% or 100%. In some embodiments, the activity of a liver isoform of CPT1 (CPT1L) is reduced. In one embodiment, CPT1L is preferentially reduced relative to other CPT1 enzymes.

As used herein the term “disease or disorder associated with carnitine palmitoyl transferase” refers to a disease or disorder in which a decrease in CPT activity would provide a beneficial (e.g., therapeutic) effect. The disease or disorder may be one that is at least in part due to excess activity of CPT1, such as a disease or disorder that is directly related to the catalysis of long-chain acylcarnitines by CPT1. Alternatively, the disease or disorder may be one that is indirectly related to the activity of CPT1, such as a disease or disorder having a symptom that is treatable by decreasing levels of CPT1 activity.

In a further aspect, the invention provides methods of treating a disease or disorder by administering an effective amount of a compound having Formula I or II to a subject in need thereof. In particular embodiments, the disease or disorder may be a metabolic disorder such as diabetes mellitus (e.g., type I or type II), metabolic syndrome, hyperglycemia, insulin resistance, glucose intolerance and/or obesity. In other embodiments, the disease or disorder is leptin resistance, gonadotropin deficiency, heart failure, ischemia, atherosclerosis, coronary artery disease, hyperlipidemia, hypertriglyceridemia, hypercholesterolemia, hypertension, familial lipoprotein deficiency, amenorrhea, and/or polycystic ovary syndrome. In a further embodiment, the disease or disorder is a hyperproliferative disease, such as cancer, e.g., lung, colon, prostate, breast, brain, head and neck, ovarian, uterine, or testicular cancer, leukemia, or lymphoma. In another embodiment, the disease or disorder is a skin disorder, including without limitation, psoriasis, acne, actinic keratosis, atopic dermatitis, dermatomyositis, rosacea, urticaria, angioedema, seborrheic dermatitis, cutaneous atopy (e.g., eczema), Darrier's disease, xerosis, ichtyosis, pigmentation disorders, hyperkeratosis, mycosis fungoides, lichen planus, and hyperplasia of the epidermis. When referring to a skin disorder, the word “skin” is meant to include any layer(s) of the skin in which a skin disorder may occur, extend to and/or reside, including that on limbs, trunk, head, as well as mucosa, etc. Thus, the word “skin” is intended to include, but not be limited to, the epidermal and/or dermal layers, and may also include the underlying subcutaneous tissue.

As used herein, an “effective amount” refers to an amount of a compound that is sufficient to produce a desired effect, which is optionally a therapeutic effect (i.e., by administration of a therapeutically effective amount). For example, an “effective amount” can be an amount that is sufficient to treat a disease or disorder such as diabetes or psoriasis. In one embodiment, an effective amount is an amount that decreases CPT1 activity by at least about 10%, e.g., at least about 20%, 30%, 40%, 50%, 60%, 70%, 80%, 90% or more. In another embodiment, an “effective amount” can be an amount that is sufficient to improve at least one symptom of a disease or disorder.

As used herein, the terms “treat,” “treating,” and “treatment” refer to any type of action that imparts a modulating effect, which, for example, can be a beneficial effect, to a subject afflicted with a disorder, disease or illness, including improvement in the condition of the subject (e.g., in one or more symptoms), delay or reduction in the progression of the condition, prevention or delay of the onset of the disorder, and/or change in clinical parameters, disease or illness, etc., as would be well known in the art.

CPT1 activity can be measured by methods well known in the art, such as spectrophotometric or chromatographic assays. Examples of CPT1 assays include those described in U.S. Pat. No. 6,369,073 and in Kerner et al., Biochemistry 29:4326 (1990), each herein incorporated by reference. In one embodiment, the effect of compounds on CPT1 activity is assayed in Wistar rat liver mitochondria. Compounds are added to mitochondrial membranes and substrate (20 μM L-carnitine+L-[methyl-¹⁴C]-carnitine) in the presence of buffer (131.25 mM Tris-HCl, pH 7.4, 0.31 mM reduced glutathione, 5 mM ATP, 5 mM MgCl₂, 18.75 mM KCl, 0.005% rotenone, 1.25% BSA) and incubated for 10 min at 37 C . Activity is measured by quantitation of [¹⁴C]-palmitoylcarnitine.

A “therapeutically effective” amount as used herein is an amount that provides some improvement or benefit to the subject (e.g., reduction of blood glucose levels in a diabetic patient or a decrease in severity of psoriasis). Alternatively stated, a “therapeutically effective” amount is an amount that provides some alleviation, mitigation, delay and/or decrease in at least one clinical symptom and/or prevents the onset or progression of at least one clinical symptom. Clinical symptoms associated with the diseases or disorders that can be treated by the methods of the invention are well-known to those skilled in the art. Further, those skilled in the art will appreciate that the therapeutic effects need not be complete or curative, as long as some benefit is provided to the subject.

In one embodiment, more than one compound of the invention is administered to a subject, e.g., 2, 3, 4, or more compounds. In another embodiment, a compound of the invention is administered to a subject concurrently with another therapeutic agent, e.g., any agent that is known to be effective for treating a disease or disorder. As used herein, the word “concurrently” means sufficiently close in time to produce a combined effect (that is, concurrently may be simultaneously, or it may be two or more events occurring within a short time period before or after each other). The other agents may be administered separately from the compounds of the present invention, or the two combined together in a single composition.

In one embodiment, the therapeutic agent is one that is useful in the treatment of a skin disorder. For example, the compounds of the invention can be administered in conjunction with an inhibitor of malonyl CoA, anti-inflammatory agents including steroids and/or non-steroidal compounds, a local anesthetic, other inhibitors of fatty acid oxidation (e.g., malonyl CoA decarboxylase inhibitors or other CPT1 inhibitors), vitamin D analogues (e.g., calcipotriene), Infliximab, Adalimumab, Etanercept, Alefacept, Efalizumab, immunosuppressants (e.g., tacrolimus), phosphodiesterase-IV inhibitors (e.g., CC-10004), JB-991, AN-0128, AN-2728, a retinoid (e.g., tazarotene), anthralin, salicylic acid, an anti-IL12 antibody, an anti-IL23 antibody, an anti-IL15 antibody, coal tar, dithranol, urea, Mahonia aquifolium, vitamin B or derivatives thereof (e.g., vitamin B12), antibiotics, antimycotics, immunomodulators (e.g., methotrexate, cyclosporine), and/or systemic treatment with fumaric acid, fumaric acid esters and/or blockers of arachidonic acid (e.g., omega-3 fatty acids).

In one embodiment, the compounds of the invention are administered in conjunction with anti-cancer agents, such as 1) vinca alkaloids (e.g., vinblastine, vincristine); 2) epipodophyllotoxins (e.g., etoposide and teniposide); 3) antibiotics (e.g., dactinomycin (actinomycin D), daunorubicin (daunomycin; rubidomycin), doxorubicin, bleomycin, plicamycin (mithramycin), and mitomycin (mitomycin C)); 4) enzymes (e.g., L-asparaginase); 5) biological response modifiers (e.g., interferon-alfa); 6) platinum coordinating complexes (e.g., cisplatin and carboplatin); 7) anthracenediones (e.g., mitoxantrone); 8) substituted ureas (e.g., hydroxyurea); 9) methylhydrazine derivatives (e.g., procarbazine (N-methylhydrazine; MIH)); 10) taxanes (e.g., paclitaxel, docetaxel); 11) adrenocortical suppressants (e.g., mitotane (o,p′-DDD) and aminoglutethimide); 12) adrenocorticosteroids (e.g., prednisone); 13) progestins (e.g., hydroxyprogesterone caproate, medroxyprogesterone acetate, and megestrol acetate); 14) estrogens (e.g., diethylstilbestrol and ethinyl estradiol); 15) antiestrogens (e.g., tamoxifen); 16) androgens (e.g., testosterone propionate and fluoxymesterone); 17) antiandrogens (e.g., flutamide): and 18) gonadotropin-releasing hormone analogs (e.g., leuprolide).

In another embodiment, the compounds of the invention are administered in conjunction with anti-diabetes agents, such as insulin, insulin agonists, insulin-like growth factor (IGF), IGF agonists, biguanides (such as metformin (GLUCOPHAGE)), thiazolidinediones (such as rosiglitazone (AVANDIA), pioglitazone (ACTOS), troglitazone (REZULIN), englitazone, and ciglitazone), MBX-102 (an enantiomer of halogenate), insulin secretagogues, including meglitinides (such as repaglinide (PRANDIN) and nateglinide (STARLIX)), sulfonylureas (such as tolbutamide, chlorpropamide (DIABINASE), tolazamide (TOLINASE), glyburide (MICRONASE, DIABETA), glypizide (GLUCOTROL), and glimepiride (AMARYL)), and alpha-glucosidase inhibitors (such as acarbose (PRECOSE) and miglitol (GLYSET)). Other useful agents include peroxisome proliferator-activated receptor (PPAR) agonists, including selective agonists of PPAR-α, PPAR-γ, and PPAR-δ, as disclosed in U.S. Pat. Nos. 6,713,514, 6,677,298, 6,462,046, 5,925,657, and 5,326,770 and in Combs et al., J. Neurosci. 20:558 (2000). Useful PPAR-δ receptor selective agonists include without limitation GW 501516, GW 0742, L-165041, and carbaprostacyclin.

The present invention finds use in research as well as veterinary and medical applications. The term “animal” as used herein includes both avians and mammals. The term “avian” as used herein includes, but is not limited to, chickens, ducks, geese, quail, turkeys and pheasants. The term “mammal” as used herein includes, but is not limited to, humans, non-human primates, cattle, sheep, goats, pigs, horses, cats, dog, rabbits, rodents (e.g., rats and/or mice), etc. In particular embodiments, the subject is a human subject that has been diagnosed with or is considered at risk for a disease or disorder, e.g., one that is associated with carnitine palmitoyl transferase.

The subject can be a subject that has been diagnosed with or is at risk for a disease or disorder, e.g., one that is associated with carnitine palmitoyl transferase. Human subjects include neonates, infants, juveniles, and adults. In other embodiments, the subject used in the methods of the invention is an animal model of a disease or disorder, e.g., one associated with carnitine palmitoyl transferase. The compounds of the invention can be used to study diseases and disorders, e.g., ones that are associated with CPT, in animal models of disease, as well as to study the function of CPT in isolated cells or cell lines, and in solution.

The compounds of the invention may be formulated for administration in a pharmaceutical carrier in accordance with known techniques. See, e.g., Remington, The Science And Practice of Pharmacy (9^{th} Ed. 1995). In the manufacture of a pharmaceutical formulation according to the invention, the compound is typically admixed with, inter alia, an acceptable carrier. The carrier must, of course, be acceptable in the sense of being compatible with any other ingredient in the formulation and must not be deleterious to the patient. The carrier may be a solid or a liquid, or both, and may be formulated with the compound as a unit-dose formulation, for example, a tablet, which may contain from 0.01 or 0.5% to 95% or 99% by weight of the compound. One or more compound may be incorporated in the formulations of the invention, which may be prepared by any of the well known techniques of pharmacy comprising admixing the components, optionally including one or more accessory ingredients.

The formulations of the invention include those suitable for oral, rectal, buccal (e.g., sub-lingual), vaginal, parenteral (e.g., subcutaneous, intramuscular, intradermal, or intravenous), topical (i.e., skin and/or mucosal surfaces, including airway surfaces) and transdermal administration, although the most suitable route in any given case will depend on the nature and severity of the condition being treated and on the nature of the particular active compound which is being used.

Formulations suitable for oral administration may be presented in discrete units, such as capsules, cachets, lozenges, or tablets, each containing a predetermined amount of the active compound; as a powder or granules; as a solution or a suspension in an aqueous or non-aqueous liquid; or as an oil-in-water or water-in-oil emulsion. Such formulations may be prepared by any suitable method of pharmacy which includes the step of bringing into association the compound and a suitable carrier (which may contain one or more accessory ingredients as noted above). In general, the formulations of the invention are prepared by uniformly and intimately admixing the compound with a liquid or finely divided solid carrier, or both, and then, if necessary, shaping the resulting mixture. For example, a tablet may be prepared by compressing or molding a powder or granules containing the compound, optionally with one or more accessory ingredients. Compressed tablets may be prepared by compressing, in a suitable machine, the compound in a free-flowing form, such as a powder or granules optionally mixed with a binder, lubricant, inert diluent, and/or surface active/dispersing agent(s). Molded tablets may be made by molding, in a suitable machine, the powdered compound moistened with an inert liquid binder.

Formulations suitable for buccal (sub-lingual) administration include lozenges comprising the compound in a flavored base, usually sucrose and acacia or tragacanth; and pastilles comprising the compound in an inert base such as gelatin and glycerin or sucrose and acacia.

Formulations of the present invention suitable for parenteral administration comprise sterile aqueous and non-aqueous injection solutions of the compound, which preparations are preferably isotonic with the blood of the intended recipient. These preparations may contain anti-oxidants, buffers, bacteriostats and solutes which render the formulation isotonic with the blood of the intended recipient. Aqueous and non-aqueous sterile suspensions may include suspending agents and thickening agents. The formulations may be presented in unit\dose or multi-dose containers, for example sealed ampoules and vials, and may be stored in a freeze-dried (lyophilized) condition requiring only the addition of the sterile liquid carrier, for example, saline or water-for-injection immediately prior to use. Extemporaneous injection solutions and suspensions may be prepared from sterile powders, granules and tablets of the kind previously described. For example, in one aspect of the present invention, there is provided an injectable, stable, sterile composition comprising one or more compounds, in a unit dosage form in a sealed container. The compound is provided in the form of a lyophilizate which is capable of being reconstituted with a suitable pharmaceutically acceptable carrier to form a liquid composition suitable for injection thereof into a subject. The unit dosage form typically comprises from about 0.1 mg to about 10 grams of the compound. When the compound is substantially water-insoluble (e.g., when conjugated to a lipid), a sufficient amount of emulsifying agent which is physiologically acceptable may be employed in sufficient quantity to emulsify the compound in an aqueous carrier. One such useful emulsifying agent is phosphatidyl choline.

Formulations suitable for rectal administration are preferably presented as unit dose suppositories. These may be prepared by admixing the compound with one or more conventional solid carriers, for example, cocoa butter, and then shaping the resulting mixture.

The compounds may be administered topically and can be formulated for topical administration in a pharmaceutical carrier in accordance with known techniques. See, e.g., Remington, The Science And Practice of Pharmacy (20^{th} edition, 2000). Suitable nontoxic pharmaceutically acceptable topical carriers will be apparent to those skilled in the art of topical pharmaceutical formulations (see, e.g., Remington's Pharmaceutical Sciences (Maack Publishing Co., Easton latest edition). Further, it will be understood by those skilled in the art that the choice of suitable carriers, absorption enhancers, humectants, adhesives, etc., will typically depend on the nature of the active compound and the particular topical formulation.

Topical formulations are known in the art. Suitable pharmaceutical compositions for topical administration include, but are not limited to, a lotion, liquid, cream, ointment, salve, emulsion, milk, powder, impregnated pad, solution, spray, suspension or gel. Further, the pharmaceutical composition can take the form of a shampoo, conditioner, hair tonic, hair spray, or hair foam. The active compound may be present as a suspension or a solution. Carriers which may be used include petroleum jelly, lanoline, polyethylene glycols, alcohols, transdermal enhancers, and combinations of two or more thereof.

The active compound can optionally be formulated for extended and/or controlled release as is known in the art, e.g. as lipid or polymeric microspheres or nanospheres or vesicles, or a polymeric patch or hydrogel. Depending on the treatment specification for the disease or disorder, the optimal formula will have good skin penetration and dermal deposition to deliver an effective amount of the formulation.

Formulations suitable for transdermal administration may be presented as discrete patches adapted to remain in intimate contact with the epidermis of the recipient for a prolonged period of time. Formulations suitable for transdermal administration may also be delivered by iontophoresis (see, for example, Pharm. Res. 3:318 (1986)) and typically take the form of an optionally buffered aqueous solution of the compound. Suitable formulations comprise citrate or bis\tris buffer (pH 6) or ethanol/water and contain from 0.1 to 0.2 M active ingredient.

Compounds of the present invention may also be administered to the olfactory and/or sinus region. Delivery of the compound may be in the form of nasal drops, a nasal spray, or an aerosol.

Further, the present invention provides liposomal formulations of the compounds disclosed herein. The technology for forming liposomal suspensions is well known in the art. When the compound is in the form of an aqueous-soluble material, using conventional liposome technology, the same may be incorporated into lipid vesicles. In such an instance, due to the water solubility of the compound, the compound will be substantially entrained within the hydrophilic center or core of the liposomes. The lipid layer employed may be of any conventional composition and may either contain cholesterol or may be cholesterol-free. When the compound of interest is water-insoluble, again employing conventional liposome formation technology, the compound may be substantially entrained within the hydrophobic lipid bilayer which forms the structure of the liposome. If the compound is amphipathic in nature, the compound may be situated such that the lipophilic portion of the molecule is entrained in the lipid bilayer of the liposome and the hydrophilic portion of the molecule extends from the external and/or internal surface of the bilayer. In any instance, the liposomes which are produced may be reduced in size, as through the use of standard sonication and homogenization techniques.

Of course, the liposomal formulations containing the compound disclosed herein, may be lyophilized to produce a lyophilizate which may be reconstituted with a pharmaceutically acceptable carrier, such as water, to regenerate a liposomal suspension.

Other pharmaceutical compositions may be prepared from the compounds disclosed herein, such as aqueous base emulsions. In such an instance, the composition will contain a sufficient amount of pharmaceutically acceptable emulsifying agent to emulsify the desired amount of the compound. Particularly useful emulsifying agents include phosphatidyl cholines and lecithin.

In addition to compound, the pharmaceutical compositions may contain other additives, such as pH-adjusting additives. In particular, useful pH-adjusting agents include acids, such as hydrochloric acid, bases or buffers, such as sodium lactate, sodium acetate, sodium phosphate, sodium citrate, sodium borate, or sodium gluconate. Further, the compositions may contain microbial preservatives. Useful microbial preservatives include methylparaben, propylparaben, and benzyl alcohol. The microbial preservative is typically employed when the formulation is placed in a vial designed for multidose use. Of course, as indicated, the pharmaceutical compositions of the present invention may be lyophilized using techniques well known in the art.

The therapeutically effective dosage of any one compound, the use of which is in the scope of present invention, will vary somewhat from compound to compound, and patient to patient, and will depend upon factors such as the age and condition of the patient and the route of delivery. Such dosages can be determined in accordance with routine pharmacological procedures known to those skilled in the art. As a general proposition, a dosage from about 0.001 or 0.01 to about 250 or 500 mg/kg will have therapeutic efficacy, with all weights being calculated based upon the weight of the active compound, including the cases where a salt is employed. A dosage from about 1 mg/kg to about 200 mg/kg may be employed for oral administration. Typically, a dosage from about 0.1 mg/kg to 100 mg/kg may be employed for intramuscular injection. For topical administration, a dosage of about 0.001% to about 50% w/w active compound in a topically-acceptable medium, e.g., about 0.01% to about 10%, may be employed. For acute conditions, the duration of the treatment is usually once per day for a period of two to three weeks or until the condition is essentially controlled. For chronic conditions, the duration of treatment can be for a period of 1, 2, 3, 4, 5, or 6 months or longer, or indefinitely as needed. The treatment may be administered more frequently than once per day (e.g., 2, 3, or 4 times per day) or less frequently than once per day (e.g., once every 2, 3, 4, 5, or 6 days or once every 1, 2, 3, or 4 weeks). The treatment may be for a period of time (e.g., 1, 2, 3, or 4 weeks or more) followed by a period in which treatment is not given (e.g., 1, 2, 3, or 4 weeks or more) and then treatment is started again. This pattern, or variations of it, may be repeated as needed. Lower doses given less frequently can be used prophylactically to prevent or reduce the incidence of recurrence of the disease.

Another aspect of the invention relates to kits comprising the compounds of the invention. The kits may comprise the compounds themselves or pharmaceutical compositions comprising the compounds. The kits may comprise a single compound or two or more different compounds in separate containers and/or pooled in one container. The kits may further comprise other components for use with the compounds of the invention. Examples of other components include, without limitation, other therapeutic agents, buffers, solutions, syringes, etc. The kits may comprise a carrier, package and/or container that is compartmentalized to receive one or more containers such as vials, tubes, and the like, each of the container(s) comprising one of the separate elements.

The present invention is explained in greater detail in the following non-limiting Examples.

### EXAMPLE 1

### Preparation of (R)-3-Aminocarnitine Isobutyl Ester Chloride Hydrochloride

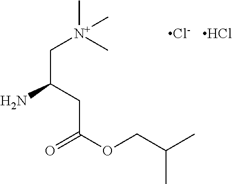

(R)-3-Aminocarnitine chloride hydrochloride (5.1 g, 21.89 mmol) was treated with Dowex 550A (OH) resin in methanol for 30 minutes. The resin was removed by filtration and the solution of free base concentrated to give a colorless syrup in essentially quantitative yield. The free base was redissolved in isobutanol (100 mL). Thionyl chloride (9.8 mL, 131.33 mmol) was added dropwise, whereupon the mixture was heated to 90° C. for 14 h An absorption train for sequestering evolved hydrogen chloride and sulphur dioxide was required. The solvent was removed under vacuum. Addition of ether to the resulting pale yellow oil caused the immediate precipitation of a colorless solid. This was collected under a flowing stream of nitrogen and dried under vacuum. The resulting hygroscopic powder (6.04 g, 96%) was stored in a stoppered vessel under an argon atmosphere. LCMS: Rt=0.985 (m/z=217). ¹H NMR (400 MHz, D₂O): 4.20 (m, 1H), 3.83 (d J=6.55 Hz, 2H), 3.73 (dd, J=4.55, 1.77 Hz, 2H), 3.12 (s, 9H), 2.96-2.87 (m, 2H), 1.80 (septet, J=6.82 Hz, 1H), 0.76 (d, J=6.82 Hz, 6H).

### EXAMPLE 2

### Preparation of Isobutyl (R)-4-Trimethylammonio-3-[3-[6-(4-phenylbutoxy)hex-1-yl]ureido]butyrate formate

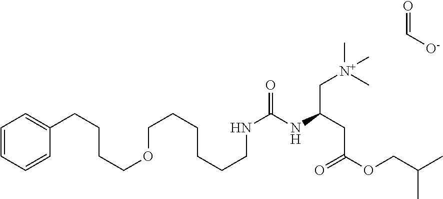

### Preparation of the Intermediate 6-(4-phenylbutoxy)hexanenitrile

Sodium hydride (60% in oil, 480 mg, 1.2 mmol) was suspended in N,N-dimethylformamide (10 mL) with ice cooling under N₂. 4-Phenyl-1-butanol (1.54 mL, 1 mmol) was added over approx. 5 minutes. After stirring for a further 30 minutes, 6-bromohexanenitrile (1.59 mL, 1.2 mmol) was introduced dropwise. Following complete addition, the mixture was allowed to come to room temperature and was stirred for 19 h. Water (50 mL) was added. When suspended solids had dissolved, the solution was extracted with ethyl acetate (3×25 mL). The combined organic phases were dried (MgSO₄), filtered and evaporated to give a pale, yellow free flowing oil. Chromatography over SiO₂ (isohexane-20% ethyl acetate/isohexane gradient) gave 6-(4-phenylbutoxy)hexanenitrile as a colorless oil (973 mg, 40%). LCMS: Rt=3.35 min, m/z=268.06 (MNa⁺), 245.95 (MH⁺). ¹H NMR (400 MHz, CDCl₃): 7.20 (m, 2 H), 7.10 (m, 3H), 3.31 (app dd, J=6.3, 5.6 Hz, 4H), 2.81 (app t, J=7.33 Hz, 2H), 2.23 (app t, J=7.07 Hz, 2H), 1.62-1.40 (m, 10H).

Preparation of the Intermediate 6-(4-phenylbutoxy)hexylamine

6-(4-Phenylbutoxy)hexanenitrile (1.76 g, 7.18 mmol) was dissolved in THF (20 mL) and added dropwise under N₂ to a cooled, stirred suspension of lithium aluminum hydride (0.75 g, 19.75 mmol) in the same solvent (10 mL). When addition was complete, the mixture was heated to 50° C. for 16 h, and then cooled in ice/water. 0.5M Potassium hydroxide solution (20 mL) was added cautiously until effervescence subsided, after which the remaining solution was run in continuously. The resulting milky suspension was stirred at room temperature for 2 h then filtered through Celite (occasional stirring required to avoid compaction of insolubles). The filtrate was acidified with 2N hydrochloric acid and extracted with ethyl acetate (2×20 ml). The aqueous phase was made basic by the addition of 2N NaOH and extracted further with ethyl acetate (5×20 mL). These extracts were dried (MgSO4), filtered and evaporated to give a pale yellow oil, 0.53 g (31%). ¹H NMR (400 MHz, CDCl₃): 7.23-7.16 (m, 2H), 7.13-7.06 (m, 3H), 3.36-3.26 (m, 4H), 2.92-2.82 (m, 1H), 2.60-2.48 (m, 2H), 1.73-1.65 (m, 1H), 1.62-1.42 (m, 7H), 1.32-1.26 (m, 3H).

Preparation of the Intermediate 6-(4-phenylbutoxy)hexyl Isocyanate

To a solution of triphosgene (0.177 g, 0.396 mmol) in anhydrous dichloromethane (5 mL) was added dropwise over 30 minutes a solution of 6-(4-phenylbutoxy)hexylamine (0.266 g, 1.069 mmol) and DIPEA (0.409 mL, 2.35 mmol) in the same solvent (2.5 mL). Following complete addition, the mixture was stirred at room temperature for lh then the solvent removed under vacuum. The residue was treated with ether (10 mL) for 30 minutes. Insoluble solids were removed by filtration and washed with ether. The combined filtrate and washings were concentrated under vacuum, giving the title compound as a colorless oil (0.170 g, 58%) which was carried forward directly. ¹H NMR (400 MHz, CDCl₃): 7.23-7.17 (m, 2H), 7.13-7.07 (m, 3H), 3.37-3.26 (m, 4H), 2.90-2.84 (m, 1H), 2.60-2.52 (m, 2H), 1.73-1.65 (m, 1H), 1.63-1.43 (m, 8H), 1.35-1.26 (m, 4H).

### Preparation of Isobutyl (R)-4-Trimethylammonio-3-[3-[6-(4-phenylbutoxy)hex-1-yl]ureido]butyrate Formate

The crude isocyanate (170 mg, 0.618 mmol), (R)-3-aminocarnitine isobutyl ester chloride hydrochloride (268 mg, 0.927 mmol) and DIPEA (236 μL, 1.360 mmol) were combined in isobutanol (5 mL) at RT and stirred for approx. 24 h. The solvent was removed under vacuum. The residue was taken up in aqueous acetonitrile and subjected to preparative HPLC. Appropriate fractions were combined and evaporated to give the title compound as a colorless oil, 22 mg (7%). ¹H NMR (400 MHz, CDCl₃): 7.23-7.17 (m, 2H), 7.13-7.09 (m, 3H), 4.77-4.56 (m, 1H), 4.34-4.25 (m, 2H), 3.79 (dd, J=10.60, 6.53 Hz, 1H), 3.73 (dd, J=10.60, 6.59 Hz, 1H), 3.33 (t, J=6.57 Hz, 2H), 3.30 (t, J=6.57 Hz, 2H), 3.20 (s, 9H), 3.12-3.00 (m, 2H), 2.74-2.53 (m, 4H), 1.84 (septet, J=6.57 Hz, 1H), 1.65-1.37 (m, 8H), 1.30-1.20 (m, 4H), 0.84 (d, J=6.85 Hz, 61-1).

### EXAMPLE 3

### Preparation of (R)-4-Trimethylammonio-3-[3-[6-(4-phenylbutoxy)hex-1-yl]ureido]butyrate Inner Salt

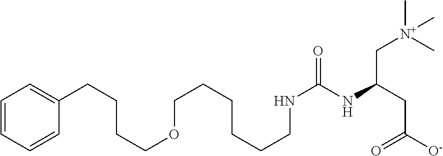

Isobutyl (R)-4-trimethylammonio-3-[3-[6-(4-phenylbutoxy)hex-1-yl]ureido]butyrate formate (22 mg, 0.042 mmol) (from Example 2) and Dowex 550A (OH) (200 mg) were combined in isobutanol and stirred at room temperature for 38 h. The resin was removed by filtration and washed with further isobutanol (5 mL). The combined filtrate and washings were evaporated and the residue taken up in 50% aqueous acetonitrile. Lyophilization for ca. 16 h gave a colorless gum, 8.2 mg (45%). LCMS: Rt=1.91 min (m/z=871.25, 456.13, 377.10). ¹H NMR (400 MHz, CD₃OD): 7.17-7.12 (m, 2H), 7.09-7.02 (m, 3H), 4.45-4.40 (m, 1H), 3.50 (dd, J=13.60, 9.35 Hz, 1H), 3.33 (t, J=6.32 Hz, 2H, 3.30 (t, J=6.57 Hz, 2H), 3.08 (s, 9H), 3.00 (t, J=7.07 Hz, 2H), 2.53 (t, J=7.33 Hz, 2H), 2.33-2.30 (m, 2H), 1.61-1.18 (m, 14H).

### EXAMPLE 4

### Preparation of (R)-4-trimethylammonio-3-[3-[6-(2-phenylethoxy)hex-1-yl]ureido]butyrate Inner Salt

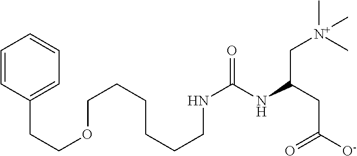

### Preparation of the intermediate 6-(2-phenylethoxy)hexanitrile

Sodium hydride (60% in oil, 1.38 g, 34.38 mmol) was suspended in N,N-dimethylformamide (45 mL) with ice cooling under N₂. 2-Phenylethanol (3.43 mL, 28.65 mmol) was added over approx. 5 minutes. After stirring for a further 30 minutes, 6-bromohexanenitrile (4.56 mL, 34.38 mmol) was introduced dropwise. Following complete addition, the mixture was allowed to come to room temperature and was stirred for 19 h. Thereafter it was heated to 50° C. for 3 h, then at room temperature for an additional 18 h. Water (200 mL) and ethyl acetate (200 mL) were added. The phases were separated and the aqueous phase extracted further with ethyl acetate (2×100 mL). The combined organic phases were dried (MgSO₄), filtered and evaporated to give a pale, yellow free flowing oil. Chromatography over SiO₂ (isohexane-20% ethyl acetate/isohexane gradient) gave the title compound as a colorless oil (1.55 g, 25%). LCMS: Rt=3.01 min, m/z=217.92, 154.67, 145.79. ¹H NMR (400 MHz, CDCl₃): 7.24-7.11 (m, 5H), 3.55 (t, J=7.07 Hz, 2H), 3.37 (t, J=6.32 Hz, 2H), 3.35 (t, J=6.57 Hz, 2H), 2.80 (t, J=7.07 Hz, 2H), 2.23-2.20 (m, 3H), 1.62-1.40 (m, 10H).

### Preparation of Intermediate 6-(2-Phenylethoxy)hexylamine

6-(2-phenylethoxy)hexanitrile (1.38 g, 6.376 mmol) was dissolved in anhydrous methanol (30 mL) and cooled in ice/water Nickel chloride (0.413 g, 3.188 mmol) was added in a single portion. Sodium borohydride (1.651 g, 44.632 mmol) was added in portions (on addition of the first portion, the mixture turned black and effervesced vigorously). When addition was complete, the mixture was brought to room temperature and stirred for 3.5 h. A further 0.413 g of nickel chloride and 0.707 g of sodium borohydride were added at this point. After a further 19 h at room temperature, the mixture was diluted with methanol (50 mL) and filtered through a Celite® pad. The filter cake was washed with methanol. The combined filtrate and washings were concentrated under vacuum to give a pale green slurry which was treated with hydrochloric acid at room temperature for lh. After filtration through Celite, the pale green solution was treated with 30% ammonium hydroxide solution to pH9 (color change to blue at approx. pH7.5). The basic solution was extracted with dichloromethane (5×30 mL). The combined extracts were washed with brine (20 mL, sat.) then dried (Na₂SO₄), filtered and evaporated to give a pale yellow oil, 0.958 g (%).

### Preparation of the Intermediate 6-(2-Phenylethoxy)hexyl Isocyanate

To a solution of triphosgene (0.475 g, 1.60 mmol) in dichloromethane (10 mL, dry) was added dropwise over 30 minutes a solution of 6-(2-phenylethoxy)hexylamine (0.266 g, 1.069 mmol) and DIPEA (0.409 mL, 2.35 mmol) in the same solvent (2.5 mL). Following complete addition, the mixture was stirred at room temperature for 1 hour then the solvent removed under vacuum. The residue was washed successively with 1N hydrochloric acid (10 mL) and 1N sodium hydroxide (10 mL). The organic phase was dried (Na₂SO₄), filtered and evaporated to give the crude isocyanate as a yellow oil, 486 mg (91%).

### Preparation of (R)-4-trimethylammonio-3-[3-[6-(2-phenylethoxy)hex-1-yl]ureido]butyrate Inner Salt

To a solution of (R)-3-amino carnitine (233 mg, 0.957 mmol) in methanol (10 mL) was added a solution of 6-(2-phenylethoxy)hexyl isocyanate (122 mg, 0.49 mmol) in the same solvent (1 mL). The mixture was stirred at room temperature for approx. 24 h. The solvent was removed under vacuum and the residue treated with acetone (10 mL). The soluble fraction was decanted away from the insoluble materials and the solvent evaporated. The residue contained the title compound of between 60 and 80% purity as determined by ¹H NMR (CD₃OD): 7.30-7.17 (m, 5H), 4.68-4.71 (m, 1H), 3.67-3.62 (m, 2H), 3.47-3.44 (m, 2H), 3.22 (s, 9H), 3.14-3.10 (m, 2H), 2.87-2.84 (m, 2H), 2.68-2.60 (m, 2H), 1.060-1.44 (m, 4H), 1.42-1.31 (m, 4H).

### EXAMPLE 5

### Preparation of Isobutyl (R)-4-Trimethylammonio-3-[3-(12-methoxydodec-1-yl)ureido]butyrate formate

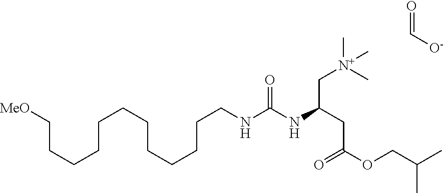

### Preparation of the Intermediate 12-Hydroxydodecanenitrile

11-Bromo-1-undecanol 5.24 g, 20.55 mmol), potassium cyanide (1.63 g, 25.05 mmol) and sodium iodide (0.63 g, 4.18 mmol) were combined in N,N-dimethylformamide (30 mL) and the mixture heated to 90° C. for approx. 48 h. The flask was cooled and the mixture diluted with water (100 mL). The resulting solution was extracted with dichloromethane (10×25 mL). The combined organics were dried (Na₂SO₄), filtered and evaporated to give an orange oil. Chromatography over SiO₂ (isohexane-20% ethyl acetate/isohexane gradient) afforded the title compound as a colorless oil, 1.31 g (33%). ¹H NMR (400 MHz, CDCl₃): 3.68 (m), 2H), 2.34 (t, J=7.07 Hz, 2H), 1.93 (b s, 1H), 1.68-1.28 (m, 18H).

### Preparation of the Intermediate 12-Methoxydodecanenitrile

12-Hydroxydodecanenitrile (1.3 g, 6.67 mmol) was dissolved in tetrahydrofuran (20 mL) and the solution cooled in ice/water. Sodium hydride (0.32 g, 8.89 mmol) was added in portions over 20 minutes. When addition was complete, the mixture was stirred at RT under N₂ for 2.5 h. Iodomethane (0.55 mL) was added in a single portion and stirring continued for a further 19 h. The crude mixture was treated with ammonium chloride (20 mL, sat.) and the phases separated. The aqueous phase was extracted further with ethyl acetate (3×10 mL) and the combined organics dried (MgSO₄), filtered and evaporated to give a yellow oil. Chromatography (isohexane-30% ethyl acetate/isohexane gradient) gave the title compound as a colorless oil, 375 mg (27%). LCMS: Rt=3.44 min (m/z=247.84, 211.93, 179.87). ¹H NMR (400 MHz, CDCl₃): 3.35 (t, J=6.57 Hz, 2H), 3.32 (s, 3H), 2.32 (t, J=7.07 Hz, 2H), 1.68-1.60 (m, 4H), 1.58-1.51 (m, 2H), 1.34-1.24 (m, 12H).

### Preparation of the Intermediate 12-Methoxydodec-1-ylamine

12-Methoxydodecanenitrile (0.375 g, 1.777 mmol) was dissolved in THF (10 mL) and added dropwise to a cooled, stirred suspension of lithium aluminum hydride (0.186 g, 4.887 mmol) in THF (10 mL). When addition was complete, the mixture was heated to 60° C. for 18 h, and then cooled in ice/water. 0.5M Potassium hydroxide solution (20 mL) was added cautiously until effervescence subsided, after which the remaining solution was run in continuously. The resulting milky suspension was stirred at room temperature for 2 h then filtered through Celite (occasional stirring required to avoid compaction of insolubles). The filter cake was washed copiously with dichloromethane. The combined filtrate and washings were partitioned and the aqueous phase extracted with dichloromethane (2×20 mL). The combined organic phases were dried (Na₂SO₄), filtered and concentrated to give the title compound as an off white solid, 264 mg (69%). LCMS (TIC): Rt=1.85 (m/z=429.20, 231.84, 215.87. ¹H NMR (400 MHz, CDCl₃): 3.29 (t, J=6.82 Hz, 2H), 3.26 (s, 3H), 2.61 (t, J=7.07 Hz), 1.52-1.45 (m, 2H), 1.39-1.32 (m, 2H), 1.25-1.15 (m, 16H).

### Preparation of the Intermediate 12-Methoxydodec-1-yl Isocyanate

To a solution of triphosgene (0.099 g, 0.332 mmol) in dichloromethane (5 mL, dry) was added dropwise over 30 minutes a solution of 12-methoxydodec-1-ylamine (0.193 g, 0.898 mmol) and DIPEA (0.343 mL, 1.975 mmol) in the same solvent (5 mL). Following complete addition, the mixture was stirred at room temperature for 1 hour then the solvent removed under vacuum. The residue was treated with ether (10 mL) for 30 minutes. Insoluble solids were removed by filtration and washed with ether. The combined filtrate and washings were concentrated under vacuum, giving the title compound as a pale yellow oil (0.161 g, 74%) which was carried forward directly. NMR (400 MHz, CDCl₃): 3.35-3.28 (m), 3.26 (s), 3.24-3.20 (m), 1.58-1.45 (m), 1.27-1.15 (m).

### Preparation of Isobutyl (R)-4-Trimethylammonio-3-[3-(12-methoxydodec-1-yl)ureido]butyrate formate

Crude 12-methoxydodec-1-yl isocyanate (0.170 mg, 0.664 mmol), (R)-3-aminocamitine isobutyl ester chloride hydrochloride (0.288 g, 0.996 mmol) and DIPEA (0.254 mL, 1.461 mmol) were combined in isobutanol (5 mL) at RT and heated to 60° C. with stirring for 19 h. The solvent was removed under vacuum. The residue was taken up in aqueous acetonitrile and subjected to preparative HPLC. Appropriate fractions were combined and evaporated to give the title compound as a colorless oil, 0.082 g (27%). NMR (400 MHz, CDCl₃): 4.72 (b s, 1H), 4.30-4.20 (m, 1H), 3.86 (dd, J=10.61, 6.57 Hz, 1H), 3.79 (dd, J=10.61, 6.57 Hz, 1H), 3.35 (t, J=6.82 Hz, 3H), 3.33 (s, 3H), 3.33 (s, 3H), 3.19-3.12 (m, 2H), 2.77-2.65 (m, 2H), 1.90 (septet, J=6.57, 1H), 1.59-1.51 (m, 2H), 1.49-1.41 (m, 2H), 1.33-1.21 (m, 2H), 0.91 (d, J=6.57Hz).

### EXAMPLE 6

### Preparation of (R)-4-Trimethylammonio-3-[3-(12-methoxy-1-dodecyl)ureido]butyrate Inner Salt

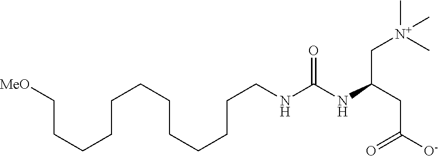

Isobutyl (R)-4-trimethylammonio-3-[3-(12-methoxy-1-dodecyl)ureido]butyrate formate (0.082 g, 0.166 mmol) (from Example 5) and Dowex 550A (OH) (0.40 g) were combined in isobutanol (5 mL) and stirred at room temperature for 38 h. The resin was removed by filtration and washed with further isobutanol (5 mL). The combined filtrate and washings were evaporated and the residue taken up in 50% aqueous acetonitrile. Lyophilization for ca. 16 h gave a colorless solid, 0.048 g (72%). LCMS: Rt=1.95 min (m/z=803.34, 402.11). ¹H NMR (400 MHz, CD₃OD): 4.43 (b s, 1H), 3.51 (dd, J=13.54, 9.35 Hz, 1H), 3.37 (dd, J=13.39, 2.02 Hz, 1H), 3.28 (t, J=6.57 Hz, 2H), 3.21 (s, 3H), 3.09 (s, 9H), 3.00 (t, J=7.07 Hz, 2H), 3.38-2.26 (m, 2H), 1.50-1.41 (m, 2H), 1.39-1.31 (m, 2H), 1.24-1.18 (m, 16H).

### EXAMPLE 7

### Preparation of Isobutyl (R)-4-Trimethylammonio-3-[3-(6-heptyloxyhex-1-yl)ureido]butyrate formate

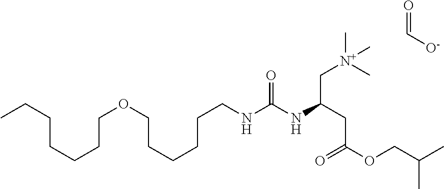

### Preparation of the Intermediate 6-Heptyloxyhexyl Isocyanate

To a solution of triphosgene (0.310 g, 1.03 mmol) in dichloromethane (20 ml, dry) was added dropwise over 30 minutes a solution of 6-heptyloxyhexylamine (0.600 g, 2.8 mmol) and DIPEA (1.07 mL, 6.14 mmol) in the same solvent (40 mL). Following complete addition, the mixture was stirred at room temperature for 1 hour then the solvent removed under vacuum. The residue was treated with ether (40 mL) for 30 minutes. Insoluble solids were removed by filtration and washed with ether. The combined filtrate and washings were concentrated under vacuum, giving the title compound as a yellow, opaque oil (0.58 g, 86%) which was processed directly.

### Preparation of Isobutyl (R)-4-Trimethylammonio-3-[3-(6-heptyloxyhex-1-yl)ureido]butyrate Formate

6-Heptyloxyhexyl isocyanate (0.58 g, 2.40 mmol), (R)-3-aminocarnitine isobutyl ester (1.04 g, 3.6 mmol) and DIPEA (0.92 mL, 5.3 mmol) were combined in isobutyl alcohol (20 mL) and stirred at room temperature for 18 hours. The solvent was removed under vacuum, leaving a pale yellow, opaque oil. Approximately one third of this residue was subjected to preparative reverse phase HPLC. Appropriate fractions were combined and evaporated, giving the title compound as a colorless oil, 0.19 g (approx. 58%). ¹H NMR (400 MHz, CD₃OD): 4.70-4.64 (m, 1H), 3.96-3.91 (m, 2H), 3.63 (dd, J=13.64, 9.85 Hz, 1H), 3.49 (dd, J=13.64, 1.52 Hz, 1H), 3.46-3.41 (m, 4H), 3.24 (s, 9H), 3.15-3.10 (m, 2H), 2.76-2.63 (m, 2H), 1.95 (septet, J=6.82 Hz, 1H), 1.62-1.53 (m, 4H), 1.52-1.46 (m, 2H), 1.42-1.29 (m, 12H), 0.97 (d, J=6.82 Hz), 0.92 (t. J=6.57 Hz, 3H).

### EXAMPLE 8

### Preparation of (R)-4-Trimethylammonio-3-[3-(6-heptyloxyhex-1-yl)ureido]butyrate

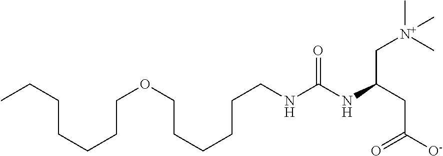

Isobutyl (R)-4-trimethylammonio-3-[3-(6-heptyloxyhex-1-yl) ureido]butyrate formate (90 mg, 0.182 mmol) (from Example 7) was dissolved in isobutyl alcohol (5 mL). Dowex 550A (OH) resin (900 mg) was added and the mixture agitated at room temperature for 16 hours. The solvent was removed under vacuum. The residue was dissolved in 50% aqueous acetonitrile (5 mL) and lyophilized for 15 h, giving the title compound as a colorless solid, 61 mg (77%). ¹H NMR (400 MHz, CD₃OD): 4.46-4.39 (m, 1H), 3.60 (dd, J=13.64, 9.60 Hz, 1H), 3.37 (dd, J=13.64, 1.77 Hz, 1H), 3.31 (app t, J=6.57 Hz, 4H), 3.09 (s, 9H), 3.01 (app t, J=6.95 Hz, 2H), 3.37-2.26, m, 2H), 1.49-1.42 (m, 4H), 1.40-1.34 (m, 2H), 1.30-1.19 (m, 12H), 0.80 (t, J=6.75 Hz, 3H).

### EXAMPLE 9

### Preparation of Isobutyl (R)-4-Trimethylammonio-3-[3-[7-(4-biphenyloxy)hept-1-yl]ureido]butyrate formate

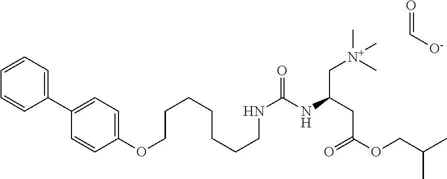

### Preparation of the Intermediate 7-(4-Biphenyloxy)heptanenitrile

4-Hydroxybiphenyl (11.1 g, 65.4 mmol) and potassium carbonate (13.6 g, 98.1 mmol) were combined in N,N-dimethylformamide (100 mL) and heated to 90° C. 7-Bromoheptanenitrile (9.8 mL, 65.4 mmol) was introduced dropwise. When addition was complete, the temperature was adjusted to 110° C. for 8 h, then to 50° C. for a further 65 h. The mixture was cooled and insoluble materials removed by filtration. The filter cake was washed with DMF and the combined filtrate and washings poured onto ice (approx. 200 g). A dense precipitate formed immediately. This was collected and dissolved in dichloromethane (500 mL). The solution was washed with brine (100 mL, sat.) then dried (Na₂SO₄), filtered and concentrated to give an off-white solid, 19.75 g (greater than theoretical; due to entrained solvent). LCMS: Rt=3.65 (m/z=296.97 (M+H₂O), 279.90 (MH⁺). ¹H NMR (400 MHz, CDCl₃): 7.50-7.41 (m, 4H 0 7.37-7.32 (m, 2H), 7.26-7.21 (m, 1H), 6.91-6.88 (m, 2H), 3.92 (t, J=6.32 Hz, 2H), 2.30 (t, J=7.07 Hz, 2H), 1.80-1.72 (m, 2H), 1.68-1.60 (m, 2H), 1.50-1.44 (m, 4H).

### Preparation of the Intermediate 7-(4-Biphenyloxy)-1-aminoheptane

7-(4-Biphenyloxy)heptanenitrile (19.70 g, assume 65.4 mmol (corresponding to theoretical recovery from previous reaction)) was dissolved in THF (200 mL) and added dropwise to a cooled, stirred suspension of lithium aluminum hydride (6.95 g, 175.2 mmol) in THF (300 mL). When addition was complete, the mixture was heated to 60° C. for 18 h, then cooled in ice/water. 0.5M Potassium hydroxide solution (200 mL) was added cautiously until effervescence subsided, after which the remaining solution was run in continuously. The resulting milky suspension was stirred at room temperature for 2 h then filtered through Celite (occasional stirring required to avoid compaction of insolubles). The filter cake was washed copiously with dichloromethane. The combined filtrate and washings were partitioned and the aqueous phase extracted with dichloromethane (2×100 mL). The combined organic phases were dried (Na₂SO₄), filtered and concentrated to give the title compound as a pale yellow solid, 12.0 g (65%). ¹H NMR (400 MHz, CDCl₃): 7.49-7.42 (m, 4H), 7.36-7.30 (m, 2H), 7.24-7.19 (m, 1H), 6.91-6.86 (m, 2H), 3.92 (t, J=6.57 Hz, 2H), 2.60 (app dd, J=7.33, 7.07 Hz) 2.62-2.57 (m, 2H), 1.77-1.69 (m, 4H), 1.45-1.37 (m, 4H).

### Preparation of the Intermediate 7-(4-Biphenyloxy)-1-heptyl Isocyanate

To a solution of triphosgene (0.110 g, 0.37 mmol) in dichloromethane (10 ml, dry) was added dropwise over 30 minutes a solution of 7-(4-biphenyloxy)-1-aminoheptane (0.283 g, 1.0 mmol) and DIPEA (0.38 mL, 2.2 mmol) in the same solvent (20 mL). Following complete addition, the mixture was stirred at room temperature for 1 hour then the solvent removed under vacuum. The residue was treated with ether (20 mL) for 30 minutes. Insoluble solids were removed by filtration and washed with ether. The combined filtrate and washings were concentrated under vacuum, giving the title compound as a yellow, opaque gum (0.106 g, 34%) which was processed directly. ¹H NMR (400 MHz, CDCl₃): 7.49-7.41 (m, 4H), 7.36-7.30 (m, 2H), 7.25-7.20 (m, 1H), 6.91-6.87 (m, 2H), 3.92 (t, J=6.57 Hz, 2H, 3.22 (t, J=6.57 Hz, 2H), 1.77-1.69 (m, 2H), 1.59-1.52 (m, 2H), 1.49-1.25 (m, 8H).

### Preparation of Isobutyl (R)-4-Trimethylammonio-3-[3-[7-(4-biphenyloxy)hept-1-yl]ureido]butyrate formate

7-(4-Biphenyloxy)-1-heptyl Isocyanate (0.088 g, 0.285 mmol), (R)-3-aminocanfitine isobutyl ester (0.123 g, 0.427 mmol) and DIPEA (0.107 mL, 0.627 mmol) were combined in isobutyl alcohol (20 mL) and stirred at room temperature for 48 hours. The solvent was removed under vacuum. The residue was dissolved in dimethyl sulphoxide (2 mL) and subjected to preparative reverse phase HPLC. Appropriate fractions were combined and evaporated to give the title compound as a colorless oil which solidified on standing, 0.042 g (28%). LCMS: Rt=2.24 min (m/z=526). ¹H NMR (400 MHz, CD₃OD): 8.47 (b s, 1H), 7.60-7.52 (m, 4H), 7.44-7.38 (m, 2H), 7.32-7.26 (m, 1H), 7.02-6.96 (m, 2H), 4.72-4.64 (m, 1H), 4.01 (t, J=6.32 Hz, 2H), 3.94-3.88 (m, 2H), 3.68-3.60 (app dd, J=13. 13, 9.60 Hz 1H), 3.49 (app d, J=13.39 Hz, 1H), 3.22 (s, 9H), 3.18-3.12 (m, 2H), 2.0-1.90 (m, 1H), 1.84-1.74 (m, 2H), 1.56-1.48 (m, 4H), 1.46-1.36 (m, 4H).

### EXAMPLE 10

### Preparation of (R)-4-Trimethylammonio-3-[3-[7-(4-biphenyloxy)hept-1-yl]ureido]butyrate Inner Salt

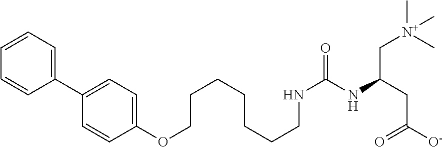

Isobutyl (R)-4-trimethylammonio-3-[3-(7-(4-biphenyloxy)hept-1-yl)ureido]butyrate formate (0.02 g, 0.038 mmol) (from Example 9) was dissolved in isobutyl alcohol (1 mL). Dowex 550A (OH) resin (0.20 g) was added and the mixture agitated at room temperature for 15 hours. The resin was removed by filtration and washed with further isobutanol (2 mL). The combined filtrate and washings were removed under vacuum. The residue was dissolved in 50% aqueous acetonitrile (5 mL) and lyophilized for 15 h, giving the title compound as a colorless solid, 9.4 mg (53%). ¹H NMR (400 MHz, CD₃OD): 7.48-7.41 (m, 4H), 7.32-7.26 (m, 2H), 7.20-7.14 (m, 1H), 6.90-6.85 (m, 2H), 4.47-4.38 (m, 1H), 3.90 (t, J=6.32 Hz, 2H), 3.50 (dd, J=13.39, 9.10 Hz, 1H), 3.38 (dd, J=13.39, 1.77 Hz, 1H), 3.09 (s, 9H), 3.02 (t, J=6.82 Hz, 2.33-2.30 (m, 2H), 1.74-1.66 (m, 2H), 1.44-1.35 (m, 4H), 1.34-1.24 (m, 4H).

### EXAMPLE 11

### Preparation of Isobutyl (R)-4-Trimethylammonio-3-[3-[7-(4-biphenylyl)hept-1-yl]ureido]butyrate formate

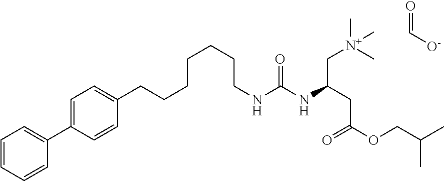

### Preparation of the Intermediate (6-Phthalimidohexyl)triphenylphosphonium Bromide

Triphenylphosphine (6.42 g, 24.50 mmol) was added to a solution of N-(6-Bromohexylphthalimide) (5.17 g, 16.67 mmol) in toluene (100 mL). The resulting clear solution was heated to reflux for approx. 18 h. The solvent was removed under vacuum and the residue treated with ether under ultrasonic irradiation for approx. 30 minutes. The resulting powder was collected by filtration and washed with ether, then dried under vacuum. Yield 3.3 g (34%). LCMS: Rt=2.06 (m/z=492.07). NB. The material is appreciably hygroscopic.

### Preparation of the Intermediate [1-(4-Biphenylyl)-7-phthalimido]-1-heptene

(6-Phthalimidohexyl)triphenylphosphonium bromide (2.5 g, 4.37 mmol) was suspended in anhydrous THF (100 mL) and cooled to −78° C. Potassium t-butoxide (0.88 g, 7.86 mmol) was added in a single portion. After 20 minutes, 4-biphenylcarboxaldehyde (0.96 g, 5.24 mmol) was added in a single portion. The mixture was allowed to come to room temperature then heated to reflux with stirring under N₂ for approx 16 h. Water (30 mL) and ethyl acetate (30 mL) were added. The aqueous phase was extracted further with ethyl acetate (2×30 mL) and the combined organic phases washed with water (30 mL) and brine (30 mL, sat.) then dried (MgSO₄), filtered and concentrated to give a yellow syrup. Chromatography over SiO₂ using an isohexane-20%ethyl acetate/isohexane gradient provided the title compound in poor yield (218 mg, 13%) and entrained with triphenylphosphine oxide. ¹H NMR (400 MHz, CDCl₃): 7.64-7.56 (m, 4H), 7.48-7.44 (m, 3H), 7.38-7.33 (m, 3H), 6.46 (dt, J=11.62, 1.52 Hz, 1H), 5.69 (dt, J=11.62, 7.07 Hz, 1H), 3.71 (app t, J=7.33 Hz, 2H), 2.40 (qd, J=7.33, 1.77 Hz, 2H), 1.78-1.68 (m, 2H), 1.60-1.51 (m, 2H), 1.47-1.39 (m, 2H).

### Preparation of the Intermediate 7-(4-Biphenylyl)-1-aminoheptane

[1-(4-Biphenylyl)-7-phthalimido]-1-heptene (218 mg, 0.554 mmol) was dissolved in ethanol (10 mL). 10% Palladium on carbon (20 mg) was added under a briskly flowing stream of nitrogen. The flask was flushed with hydrogen and then maintained under a positive pressure of the same gas with stirring for approx. 16 h. Analysis by multiple-elution TLC (10% ethyl acetate/isohexane) revealed the absence of alkene after this time. The catalyst was filtered through a pad of Celite and the pad washed with ethanol (2×5 mL). The combined filtrate and washings were treated with hydrazine monohydrate (0.26 mL) at 90° C. for 24 h. The flask was cooled and the resultant precipitate removed by filtration then washed with ethanol (10 mL). The combined filtrate and washings were concentrated under vacuum to give the title compound as a colorless solid, 144 mg (97% (contained traces of phthalazine)). LCMS: Rt=2.02 min (m/z=492.07, 268.06) (inter alia). ¹H NMR (400 MHz, CD₃OD): 7.63-7.60 (m, 2H), 7.56-7.53 (m, 2H), 7.47-7.42 (m, 3H), 7.31-7.27 (m, 2H), 2.93 (d, J=7.58 Hz, 1H), 2.9 (d, J=6.82 Hz, 1H), 2.69 (app t, J=7.58 Hz, 2H), 1.74-1.63 (m, 4H), 1.46-1.39 (m, 6H).

### Preparation of the Intermediate 7-(4-Biphenylyl)-1-heptyl Isocyanate

To a solution of triphosgene (0.074 g, 0.250 mmol) in dichloromethane (10 ml, dry) was added dropwise over 30 minutes a solution of 7-(4-biphenylyl)-1-aminoheptane (0.181 g, 0.677 mmol) and DIPEA (0.26 mL, 1.49 mmol) in the same solvent (20 mL). Following complete addition, the mixture was stirred at room temperature for 2 hours, and then the solvent removed under vacuum. The residue was treated with ether (20 mL) for 30 minutes. Insoluble solids were removed by filtration and washed with ether. The combined filtrate and washings were concentrated under vacuum, giving the title compound as a yellow oil (0.067 g, 34%) which was processed directly.

### Preparation of Isobutyl (R)-4-Trimethylammonio-3-[3-[7-(4-biphenylyl)hept-1-yl]ureido]butyrate formate

7-(4-Biphenylyl)-1-heptyl Isocyanate (0.067 g, 0.228 mmol), (R)-3-aminocarnitine isobutyl ester (0.099 g, 0.342 mmol) and DIPEA (0.087 mL, 0.502 mmol) were combined in isobutyl alcohol (5 mL) and stirred at 40° C. for 18 hours. The solvent was removed under vacuum. The residue was dissolved in dimethyl sulphoxide (2 mL) and subjected to preparative reverse phase HPLC. Appropriate fractions were combined and evaporated to give the title compound as a colorless solid, 0.021 g (18%). LCMS: Rt=2.24 min (m/z=526). ¹H NMR (400 MHz, CD₃OD): 7.60-7.57 (m, 2H), 7.53-7.49 (m, 2H), 7.44-7.38 (m, 2H), 7.32-7.27 (m, 1H), 7.26-7.23 (m, 2H), 4.68-4.61 (m, 1H), 3.93-3.85 (m, 2H), 3.58 (dd, J=13.89, 9.60 Hz, 1H), 3.46 (dd, J=13.64, 2.02 Hz, 1H), 3.18 (s, 9H), 3.10 (td, J=6.57, 1.77 Hz, 2H), 2.72-2.60 (m, 4H), 1.91 (septet, J=1.68-1.61 (m, 2H, 1.50-1.42 (m, 2H), 1.39-1.61 (m, 6H).

### EXAMPLE 12

### Preparation of (R)-4-Trimethylammonio-3-[3-[7-(4-biphenylyl)hept-1-yl]ureido]butyrate Inner Salt

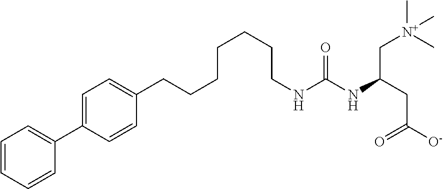

Isobutyl (R)-4-trimethylammonio-3-[3-(7-(4-biphenylyl)heptyl)ureido]butyrate formate (0.018 g, 0.032 mmol) (from Example 11) was dissolved in isobutyl alcohol (2 mL). Dowex 550A (OH) resin (0.20 g) was added and the mixture agitated at room temperature for 60 hours. The resin was removed by filtration and washed with isobutanol (2 mL). Solvent was removed under vacuum. The residue was dissolved in 50% aqueous acetonitrile (5 mL) and lyophilized for 15 h, giving the title compound as a colorless solid, 0.08 g (55%). ¹H NMR (400 MHz, CD₃OD): 7.62-7.57 (m, 2H), 7.53-7.50 (m, 3H), 7.43-7.38 (m, 2H), 7.32-7.37 (m, 1H), 7.26-7.22 (m, 2H), 4.54-4.47 (m, 1H), 3.58 (dd, J=13.64, 9.35 Hz, 1H), 3.45 (dd, J=13.64, 2.02 Hz, 1H), 3.16, (s, 9H), 3.10 (td, J=6.82, 0.76 Hz, 2H), 2.64 (dd, J=7.83, 7.32 Hz, 2H), 2.46-2.35 (m, 2H), 1.70-1.60 (m, 2H), 1.49-1.42 (m, 2H), 1.39-1.27 (m, 6H).

### EXAMPLE 13

### Skin Penetration Studies

In vitro skin penetration studies are performed on formulations containing the compounds of the present invention using Bronaugh Flow-Thru diffusion cells and/or Franz static diffusion cells. The formulations contain approximately 1 mCi of radiolabeled drug, where the radiolabel may be tritium or carbon-14. Approximately 10 to 20 grams of each formulation is provided. The formulations are tested using dermatomed abdominal human skin from a single donor. The skin is prepared to ensure uniformity and integrity. The in vitro study examines drug deposition on and in the different layers of the skin as well as drug penetration of the skin after 24 hour exposure to the formulation. After 24 hour exposure to the formulation, the residual dose from the skin surface is removed by up to three repetitive tape strips and the remaining epidermis is separated from the dermis by physical means. Analysis is performed on the diffusion cell washes, skin surface tape strips, as well as epidermis, dermis, and receptor fluid using liquid scintillation counting.

### EXAMPLE 14

### Formulation Studies

Formulations containing the compounds of the present invention are tested to determine the in vitro release of the compounds from the formulation. Approximately 20 grams of each formulation is provided. The study is conducted using a synthetic membrane (Tuffryn HT-450, 25 mm, 0.45 micron pore size) and an appropriate receptor solution. The release study runs for 6 hours using Franz static diffusion cells with an infinite dose (approximately 1.5 mL of the formulation per cell). Receptor fluid samples are collected manually at 1, 2, 3, 4, and 6 hours. The receptor fluid samples are collected, labeled, and submitted for analytical concentration determination by HPLC analysis or other appropriate methodology.

The foregoing is illustrative of the present invention, and is not to be construed as limiting thereof. The invention is defined by the following claims, with equivalents of the claims to be included therein.

1. A compound of Formula I:
RO(CH₂)ₘX (I)
wherein X is selected from the group consisting of
NHCONHCH(CH₂CO₂)⁻CH₂N(CH₃)₃⁺,
NHCONHCH(CH₂CO₂H)CH₂N(CH₃)₃⁺Y⁻, and
NHCONHCH(CH₂CO₂R¹)CH₂N(CH₃)₃⁺Y⁻;
R is selected from the group consisting of CH₃(CH₂)ₙ, PhC₆H₄(CH₂)ₚ, and Ph(CH₂)_{q};
R¹ is a C₁₋₄ straight or branched alkyl group;
Y⁻ is an anion;
m is 3 to 14;
n is 0 to 11;
p is 0 to 6;
q is 1 to 9;
wherein m plus n is 10 to 14;
m plus p is 5 to 9; and
m plus q is 8 to 12;
or pharmaceutically acceptable salts, prodrugs, or stereoisomers thereof.

2. The compound of claim 1, wherein:
R is CH₃(CH₂)ₙ;
m is 3 to 14;
n is 0 to 11; and
m plus n is 10 to 14.

3. The compound of claim 1, wherein:
R is PhC₆H₄(CH₂)ₚ;
m is 3 to 9;
p is 0 to 6; and
m plus p is 5 to 9.

4. The compound of claim 1, wherein:
R is Ph(CH₂)_{q};
m is 3 to 12;
q is 1 to 9; and
m plus q is 8 to 12.

5. The compound of claim 1, which is selected from the group consisting of:
isobutyl (R)-4-trimethylammonio-3-[3-[6-(4-phenylbutoxy)hex-1-ylureido]butyrate formate;
(R)-4-trimethylammonio-3-[3-[6-(4-phenylbutoxy)hex-1-yl]ureido]butyrate;
(R)-4-trimethylammonio-3-[3-[6-(2-phenylethoxy)hex-1-yl]ureido]butyrate;
isobutyl (R)-4-trimethylammonio-3-[3-(12-methoxydodec-1-yl)ureido]butyrate formate;
(R)-4-trimethylammonio-3-[3-(12-methoxydodec-1-yl)ureido]butyrate;
isobutyl (R)-4-trimethylammonio-3-[3-(6-heptyloxyhex-1-yl)ureido]butyrate formate;
(R)-4-trimethylammonio-3-[3-(6-heptyloxyhex-1-yl)ureido]butyrate;
isobutyl (R)-4-trimethylammonio-3-[3-[7-(4-biphenyloxy)hept-1-yl)ureido]butyrate formate;
and (R)-4-trimethylammonio-3-[3-[7-(4-biphenyloxy)hept-1-yl]ureido]butyrate;
or pharmaceutically acceptable salts, prodrugs, or stereoisomers thereof.

6. The compound of claim 1, which is selected from the group consisting of 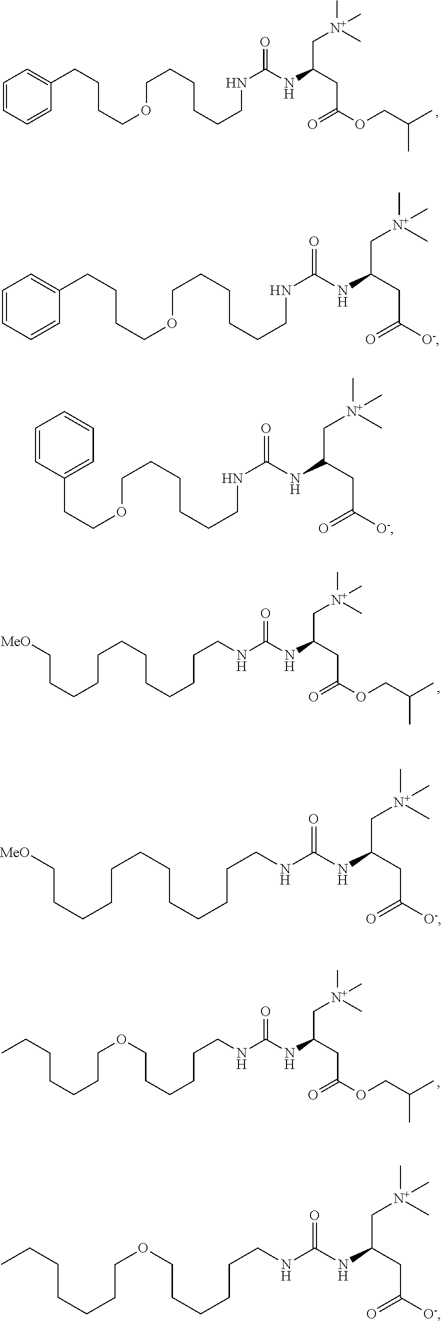 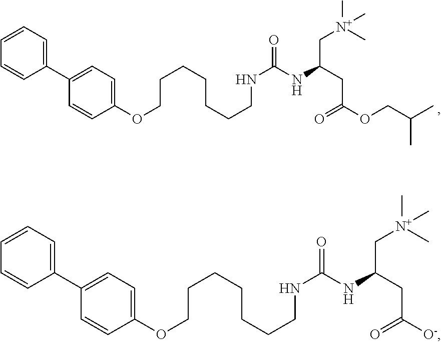
and pharmaceutically acceptable salts, prodrugs, or stereoisomers thereof.

7-14. (canceled)

15. A compound of Formula II:
R²-biphenyl(CH₂)ᵥX
wherein X is selected from the group consisting of
NHCONHCH(CH₂CO₂)⁻CH₂N(CH₃)₃⁺,
NHCONHCH(CH₂CO₂H)CH₂N(CH₃)₃⁺Y⁻, and
NHCONHCH(CH₂CO₂R¹)CH₂N(CH₃)₃⁺Y⁻;
R¹ is a C₁₋₄ straight or branched alkyl group;
Y⁻ is an anion;
R² is selected from the group consisting of H and CH₃(CH₂)_{w};
v is 2 to 10;
w is 0 to 7; and
v plus w is 5 to 10;
or pharmaceutically acceptable salts, prodrugs, or stereoisomers thereof.

16. The compound of claim 15, wherein:
R² is H; and
v is 6 to 10.

17. The compound of claim 15, wherein:
R² is CH₃(CH₂)_{w};
v is 2 to 9;
w is 0 to 7; and
v plus w is 5 to 9.

18. The compound of claim 15, which is selected from the group consisting of:
isobutyl (R)-4-trimethylammonio-3-[3-[7-(4-biphenylyl)hept-1-yl]ureido]butyrate formate and
(R)-4-trimethylammonio-3-[3-[7-(4-biphenylyl)hept-1-yl]ureido]butyrate or pharmaceutically acceptable salts, prodrugs, or stereoisomers thereof

19. The compound of claim 15, which is selected from the group consisting of: 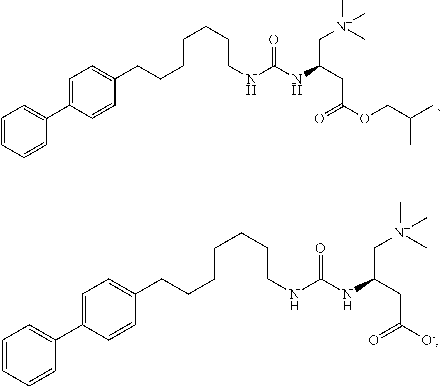
and pharmaceutically acceptable salts, prodrugs, or stereoisomers thereof

20. (canceled)

21. A pharmaceutical composition comprising a compound of claim 1 and a pharmaceutically acceptable carrier.

22. (canceled)

23. A kit comprising a compound of claim 1.

24. A method of inhibiting carnitine palmitoyl transferase 1 (CPT1) enzyme, comprising contacting the enzyme with a compound of claim 1.

25-28. (canceled)

29. A method of treating a disease or disorder in an animal, comprising administering to the animal an effective amount of a compound of claim 1.

30. The method of claim 29, wherein the disease or disorder is selected from the group consisting of leptin resistance, gonadotropin deficiency, heart failure, ischemia, atherosclerosis, coronary artery disease, hyperlipidemia, hypertriglyceridemia, hypercholesterolemia, familial lipoprotein lipase deficiency, hypertension, amenorrhea, diabetes mellitus (type I or type II), metabolic syndrome, hyperglycemia, insulin resistance, glucose intolerance, obesity, polycystic ovary syndrome, hyperphagia, skin disorders, psoriasis, atopic dermatitis, and cancer.

31-37. (canceled)

38. A process for preparing the compound of claim 1, comprising:
(a) reacting an isobutylcarnitine with a corresponding isocyanate to form an aminocarnitine-derived urea ester; and
(b) hydrolyzing the ester group of the aminocarnitine-derived urea ester to form a compound having general Formula I.

39. (canceled)

40. A pharmaceutical composition comprising a compound of claim 15 and a pharmaceutically acceptable carrier.

41. A kit comprising a compound of claim 15.

42. A method of inhibiting carnitine palmitoyl transferase 1 (CPT1) enzyme, comprising contacting the enzyme with a compound of claim 15.

43. A method of treating a disease or disorder in an animal, comprising administering to the animal an effective amount of a compound of claim 15.

44. The method of claim 43, wherein the disease or disorder is selected from the group consisting of leptin resistance, gonadotropin deficiency, heart failure, ischemia, atherosclerosis, coronary artery disease, hyperlipidemia, hypertriglyceridemia, hypercholesterolemia, familial lipoprotein lipase deficiency, hypertension, amenorrhea, diabetes mellitus (type I or type II), metabolic syndrome, hyperglycemia, insulin resistance, glucose intolerance, obesity, polycystic ovary syndrome, hyperphagia, skin disorders, psoriasis, atopic dermatitis, and cancer.

45. A process for preparing the compound of claim 15, comprising:
(a) reacting an isobutylcarnitine with a corresponding isocyanate to form an aminocarnitine-derived urea ester; and
(b) hydrolyzing the ester group of the aminocarnitine-derived urea ester to form a compound having general Formula II.
